(19) United States
(12) Patent Application Publication
(10) Pub. No.: US 2011/0293478 A1
(43) Pub. Date: Dec. 1, 2011

(54) DEVICE FOR REDUCING EVAPORATION IN A REAGENT BOTTLE

(75) Inventors: Mathieu Robert, Paris (FR); Frederic Esteve, Paris (FR); Jean-Yves Laisne, Verrieuil-En-Halatte (FR)

(73) Assignee: DIAGNOSTICA STAGO

(21) Appl. No.: 13/118,806

(22) Filed: May 31, 2011

(30) Foreign Application Priority Data
Jun. 1, 2010 (FR) 1054261

(51) Int. Cl. G01N 33/50

(52) U.S. Cl. 422/68.1

(57) ABSTRACT

A device reducing evaporation in a reagent bottle, in particular in an automatic appliance for analyzing samples, the device being in the form of a tube inserted into the bottle and including, in its top portion, an annular rim for bearing on the top end of the neck of the bottle, and means for centering in the neck of the bottle.

### FIELD OF THE INVENTION

The invention relates to a device for reducing evaporation in a reagent bottle, in particular in an automatic appliance for analyzing samples.

### BACKGROUND OF THE INVENTION

Automatic appliances for analyzing samples, in particular biological samples taken from people, make use of reagent bottles that are installed in predetermined locations, and into which automatic pipetting means are capable of inserting needles in order to take determined quantities of reagents that are then deposited it in reaction vessels containing the samples for analysis.

The reaction bottles that are installed in such appliances remain open so as to enable automatic pipetting to take place, and as a result the aqueous phase of the reagents is subjected to non-negligible amounts of evaporation, which is harmful to the stability of the reagents. Evaporation also gives rise to an increase in the concentration of the reagents in the bottles, and that runs the risk of falsifying the assays that are performed with the extracted quantities of reagent.

In order to reduce those drawbacks, proposals have been made to mount tubes in the bottles, the top ends of said tubes closing the top ends of the bottles to a greater or lesser extent, and their bottom ends enabling reagent to be sucked out from the bottles, the diameters of these tubes being smaller than the inside diameters of the bottles so that the tubes mounted in the bottles reduce the free surface areas of reagent that are exposed to atmospheric air, thereby reducing evaporation of the reagents.

Nevertheless, membrane capsules need to be fastened on the open ends of the bottles, after the tubes have been put into place, so that the bottles can be used in an automatic analysis appliance. Otherwise, the tubes are not held in the bottles and they move, running the risk of being struck by the needles of the automatic pipetting means, thereby preventing reagents being taken and damaging the pipetting means.

### SUMMARY OF THE INVENTION

An object of the invention is to provide a solution to those problems that is simple, effective, and inexpensive.

To this end, the invention proposes a device for reducing evaporation in a reagent bottle, in particular in an automatic appliance for analyzing samples, wherein the device is in the form of a tube that is open at its ends, for insertion into the reagent bottle and including, in its top portion, means for centering in the neck of the bottle, together with an annular rim bearing on the top end of the neck of the bottle.

The device of the invention presents the following advantages:

it is mounted very simply and intuitively in an open bottle;

it is held in place in the bottle as soon as it has been put into place, such that the bottle can be subjected to impacts, vibration, or movements, without the position of the device in the bottle varying; and

it is effective in reducing evaporation of the aqueous phase of the reagent, while still enabling reagent to be taken, and it greatly increases the stability of the reagent in the bottle, which bottle may remain open for several days in the analysis appliance without harm, the reduction in evaporation giving rise to reagent concentration that remains constant inside the bottle and to better control over sample assaying reactions.

In a preferred embodiment of the invention, the means for centering the tube comprise longitudinal splines formed to project from its outside surface and extending from the annular rim of the tube over a length that is substantially equal to the height of the neck of the bottle.

The bottom face of the annular rim includes bearing points for bearing against the top end of the bottle, serving to leave an empty space between the annular rim and the top end of the bottle.

By way of example, these bearing points are formed by ribs on the bottom face of the annular rim, which ribs are in alignment with the longitudinal splines for centering the tube.

By way of example, these ribs present a height lying in the range 0.2 millimeters (mm) to 0.5 mm.

According to other characteristics of the invention:

the inside diameter of the tube is less than half the inside diameter of the bottle, and for example less than or equal to about 30% of the inside diameter of the bottle;

the tube is frustoconical in shape, its smaller diameter end being its bottom end; and

the length of the tube is substantially equal to the inside height of the bottle, and is bottom end includes at least one notch for passing the reagent.

In a variant embodiment of the invention, the centering means are formed on at least two different-diameter cylindrical portions of the tube.

It is thus possible to mount the device in bottles of dimensions that differ, whether in height or in neck diameter.

The invention also provides a reagent bottle, in particular in an appliance for analyzing samples, wherein the bottle contains a device of the type described above.

### BRIEF DESCRIPTION OF THE DRAWINGS

The invention can be better understood and other details, characteristics, and advantages thereof appear more clearly on reading the following description made by way of example with reference to the accompanying drawings, in which:

FIG. 1 is a diagrammatic section view of a reagent bottle fitted with a device of the invention;

FIGS. 2 and 3 are two diagrammatic perspective views of the device of the invention; and

FIGS. 4 to 6 show a variant of the invention mounted in bottles of different sizes.

### DETAILED DESCRIPTION OF PREFERRED EMBODIMENT

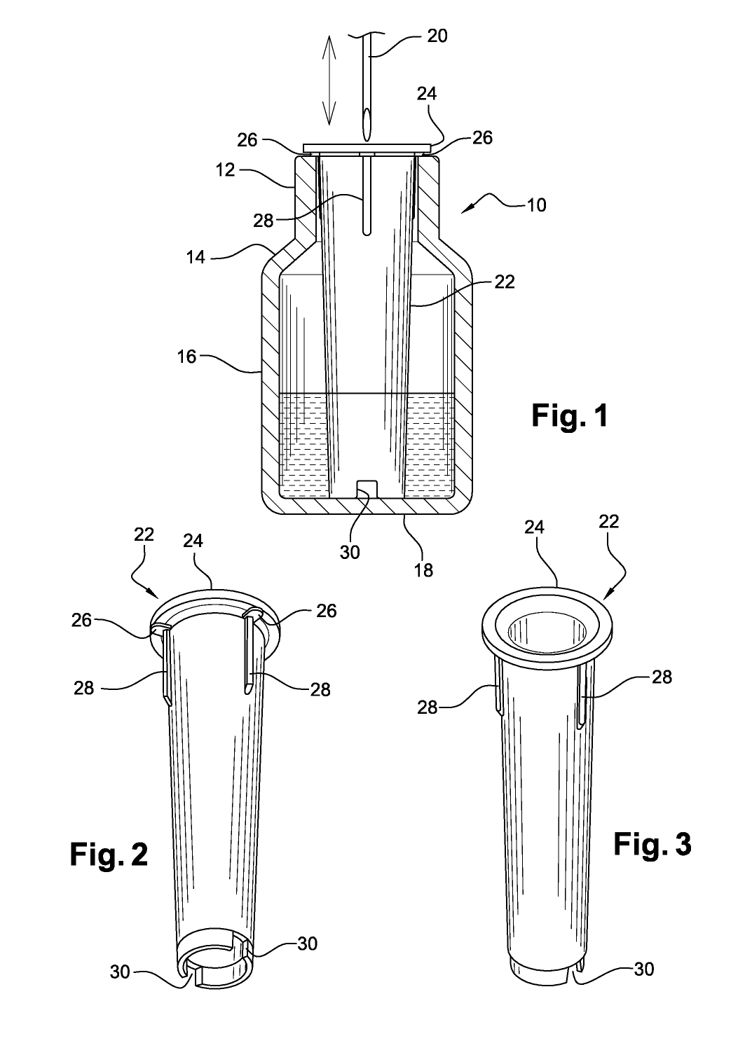

The bottle 10 shown diagrammatically in FIG. 1 is a standard bottle for containing a reagent usable in a reaction for testing a sample, in particular a sample such as a biological sample taken from a person. The bottle 10 is generally cylindrical in shape and at its top end it has a cylindrical neck 12 of smaller diameter that is connected via a frustoconical portion 14 to the cylindrical body 16 of the bottle, which bottle has a flat bottom 18.

Bottles of the type shown in FIG. 1 are generally made of glass, and the outside surface of the neck 12 of such a bottle is threaded to enable a cap to be screwed on for leaktight sealing.

The content of such bottles typically lies in the range 0 milliliters (mL) to 15 mL.

As mentioned above, these bottles are intended in particular for use in automatic sample-analyzer appliances for taking predetermined quantities of reagents by means of an automatic pipette having suction needles, one of which, 20, is shown diagrammatically in FIG. 1 above the open top end of the bottle 10.

In general, the taking of a determined quantity of reagent takes place as follows:

A suction needle 20 is brought over the bottle 10 containing the desired reagent, so as to lie on the axis of the bottle, and it is lowered into the bottle close to the bottom 18 thereof. A predetermined quantity of reagent is taken from the bottle 10 by suction, and then the needle 20 is extracted from the bottle by being moved vertically in translation, and the pipette means are placed over a reaction vessel into which the needle 20 is lowered in vertical translation so as to deposit therein the quantity of reagent it has taken. The needle 20 is then extracted from the reaction vessel and it is cleaned by washing and rinsing so as to be ready for new use.

The reagent bottles placed in the automatic analysis appliance must remain open for a certain length of time in the appliance so that sample testing can be performed in successive cycles that follow one another automatically at a relatively high rate. That inevitably leads to the evaporation of aqueous phase of the reagents contained in the bottles, which evaporation may be quite considerable, of the order of 5% to 10% per day, depending on the size of the bottles.

In order to reduce such evaporation, the invention proposes mounting a tube 22 of cylindrical or frustoconical shape in each bottle 10, the tube being open at both ends and having a top portion that includes an annular rim 24 for resting on the top end of the neck 12 of the bottle by covering a fraction of said top end, the outside diameter of the rim 24 being equal to or slightly less than the outside diameter of the neck 12 of the bottle.

The bottom face of the annular rim 24 includes projecting portions 26 that form points for bearing against the top end of the neck 12 of the bottle and that leave an empty space for controlled air flow between the top end of the neck 12 of the bottle and the annular rim 24, thereby enabling air to enter into the bottle while the reagent is being sucked up from within the tube 22 by the reagent-taking needle 20. These projecting portions are of small height, e.g. in the range 0.2 mm to 0.5 mm, approximately.

The top portion of the tube 22 includes centering means inside the neck 12 of the bottle, these centering means in this example being constituted by longitudinal splines 28 that extend from the annular rim 24 towards the bottom end of the tube 22 over a length that corresponds substantially to the height of the neck 12 of the bottle. The radial height or dimension of the projecting splines 28 on the outside surface of the tube is such that, when the tube is mounted in the bottle 10, as shown diagrammatically in FIG. 1, the tube is centered and held without slack inside the neck 12 of the bottle.

In the example shown, the outside diameter of the top portion of the tube 22 is slightly smaller than the inside diameter of the neck 12 of the bottle, and the splines 28 are of small height, of the order of 0.2 mm to 0.5 mm, for example. The bottom portion of the tube 22, which extends inside the cylindrical body 16 of the bottle, has an outside diameter that is less than half the inside diameter of the body 16 of the bottle. The thickness of the tube 22 may be relatively small, e.g. 1 mm. By way of example, the inside diameter of the tube corresponds to about ⅓rd of the inside diameter of the body 16 of the bottle, and it may be smaller than that value. This inside diameter determines the area of the free surface of the reagent contained in the tube 22 that is exposed to atmospheric air, and that is therefore subjected to evaporation. Naturally, the inside diameter of the tube 22 may be reduced down to some minimum value that allows the needle 20 to move without difficulty inside the tube 22.

The height of the tube 22 is substantially equal to the height 21 of the bottle 10, such that the bottom end of the tube 22 comes into contact with, or into the immediate vicinity of, the bottom 18 of the bottle.

In order to facilitate taking reagent from the bottle, the bottom end of the tube 20 may include one or more notches 30 through which reagent passes.

In the example shown, the tube 20 is made by injection molding a suitable plastics material, such as polypropylene, for example. It has two diametrically opposite notches 30 in its bottom end and three splines that are distributed at 120° intervals from one another at its top end, together with three bearing zones 26 that are in alignment with the splines 28.

When the bottle 10 is fitted with a tube 22 of the invention, evaporation of the aqueous phase of the reagent contained in the bottle 10 is reduced by about 60% to 70%, and the stability of the reagent over seven days in an open bottle is increased by 300% to 400%. Since the tube is accurately centered and stationary inside the neck of the bottle, reagent may be taken without problem, and it is possible to reduce the inside diameter of the tube, thereby reducing the area of the free surface of the reagent that is exposed to evaporation.

The splines 28 may present a height that is constant over their entire length, or a height that increases progressively starting from the top end of the tube, in order to compensate for the conical shape of the tube. Advantageously, they enable the tube to be lightly wedged in the neck of the bottle, such that the tube is both centered and held in the bottle.

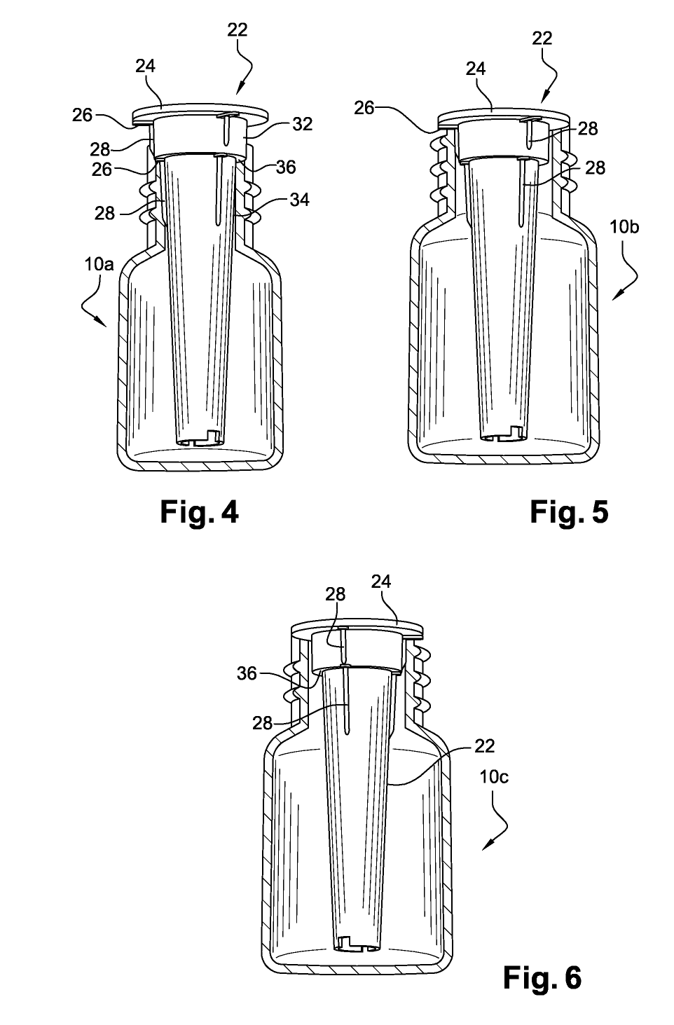

In the variant embodiment of FIGS. 4 to 6, the splines 28 are formed from the annular rim 24 on two cylindrical portions 32 and 34 of the tube that present different outside diameters, and that are connected together by a shoulder 36, the portion having a larger outside diameter being the portion connected to the annular rim 24 and the other portion possibly being constituted by the body of the tube 22. Projections 26 similar to those of the annular rim 24 are formed on the shoulder 36, for the same purpose.

It is thus possible to mount the device in bottles of different sizes, for example:

in a bottle 10a of small height and small diameter, the tube 22 bearing against the top end of the bottle via the projections 26 and the shoulder 36, and its bottom end being in the immediate vicinity of the bottom of the bottle, the tube being centered and held in the neck of the bottle by the splines 28 of its cylindrical portion 34 (FIG. 4);

in a bottle 10b of larger size, the tube 22 bearing against the top end of the bottle via the projections 26 of its annular rim 24 and its bottom end being in the immediate vicinity of the bottom of the bottle, the tube being centered and held in the neck of the bottle by the splines 28 of its cylindrical portion 32 (FIG. 5); and

in a bottle 10c of even greater size, the tube 22 bearing against the top end of the bottle via the projections 26 of its annular rim 24 and its bottom end being spaced apart from the bottom of the bottle, the tube being centered and held in the neck of the bottle by the splines 28 of its cylindrical portion 32 (FIG. 6).

Having described the invention, the following is claimed:

1. A device for reducing evaporation in a reagent bottle, in particular in an automatic appliance for analyzing samples, wherein the device is in the form of a tube that is open at its ends, for insertion into the reagent bottle and including, in its top portion, means for centering in the neck of the bottle, together with an annular rim bearing on the top end of the neck of the bottle.

2. A device according to claim 1, wherein the centering means are formed on a portion of the length of the tube, starting from the annular rim.

3. A device according to claim 1, wherein the centering means are formed on at least two cylindrical portions of the tube having different outside diameters.

4. A device according to claim 1, wherein the centering means of the tube comprise longitudinal splines formed to project from its outside surface.

5. A device according to claim 4, wherein the longitudinal splines extend from the annular rim of the tube over a length that is substantially equal to the height of the neck of the bottle.

6. A device according to claim 1, wherein the bottom face of the annular rim includes bearing points for bearing on the top end of the bottle, serving to leave an empty space between the annular rim and the top end of the bottle.

7. A device according to claim 3, wherein the two cylindrical portions of different diameters are connected together by a shoulder that includes bearing points for bearing on the top end of the bottle, and serving to leave an empty space between the shoulder and of the top end of the bottle.

8. A device according to claim 6, wherein the bearing points are formed by ribs presenting a height lying in the range 0.2 mm to 0.5 mm.

9. A device according to claim 1, wherein the inside diameter of the tube is less than half the inside diameter of the bottle.

10. A device according to claim 1, wherein the tube is frustoconical, its smaller diameter end being its bottom end.

11. A device according to claim 1, wherein the bottom end of the tube includes at least one notch for passing reagent.

12. A reagent bottle, in particular in an automatic appliance for analyzing samples, wherein the bottle contains a device according to claim 1.
